(19) United States
(12) Patent Application Publication
(10) Pub. No.: US 2025/0205084 A1
(43) Pub. Date: Jun. 26, 2025

(54) OPHTHALMIC IMPLANT WITH RESTRICTION MECHANISM ON A DRAINAGE TUBE FOR TREATING GLAUCOMA

(71) Applicant: MOLTENO OPHTHALMIC LTD

(72) Inventors: Jordan GREEN, Kent Town (AU); Victor PRIVEN, Kent Town (AU)

(73) Assignee: MOLTENO OPHTHALMIC LTD, North Dunedin (NZ)

(21) Appl. No.: 18/848,885

(22) PCT Filed: Mar. 21, 2023
(86) PCT No.: PCT/AU2023/050202

§ 371 Date: Sep. 19, 2024

(30) Foreign Application Priority Data
Mar. 21, 2022 (AU) 2022900691

(51) Int. Cl. A61F 9/007

(52) U.S. Cl. CPC A61F 9/00781; A61F 2210/0004

(57) ABSTRACT

An ophthalmic implant for treating or alleviating the symptoms of glaucoma. The implant translimbal tube is restricted to reduce the aqueous humour outflow. The implant and the restriction on the translimbal tube are sterilized together so there are no 0 further requirements for the user to undertake prior to application.

### FIELD OF THE INVENTION

The present invention relates to devices and methods for placing a restriction on a drainage tube for an ophthalmic implant. The implant along with the restriction on the drainage tube is sterilised and provided to the user ready for application on a patient.

### BACKGROUND TO THE INVENTION

Glaucoma is a term which refers to a group of eye diseases that gradually destroy vision. Vision loss due to glaucoma is caused by damage to the optic nerve. High intraocular pressure (IOP) is a risk factor for glaucoma. Glaucoma can also result in high IOP. Glaucoma implants therefore seek to reduce IOP within the eye and to maintain long-term IOP at normal levels.

The glaucoma implant of this invention is a further development and improvement on glaucoma implants invented by the same inventor which have been the subject of earlier patents.

U.S. Pat. No. 4,457,757 describes an implant that is inserted into an eye under the outer tissue layer (Tenon's tissue). The implant comprises a plate which is attached to the surface of the eyeball, and a tube that extends from the plate and is inserted into an anterior chamber of the eye. This enables aqueous fluid to be transferred, via the tube, from the anterior chamber to the plate where the fluid is absorbed into the surrounding tissue. IOP is thereby reduced.

One problem associated with implants of this kind is an excessive lowering of IOP following implantation (post-operative hypotony). This can cause damage to the eye resulting in impairment of vision.

U.S. Pat. No. 4,750,901 describes an improved implant that seeks to prevent post-operative hypotony. This implant has two elevated ridges on the upper surface of the plate. One ridge runs around the edge, or near to the edge, of the plate to help define and maintain a bleb into which fluid from the eye can drain and be absorbed into the overlaying Tenon's tissue. A subsidiary ridge defines a second smaller region within the area of the plate defined by the peripheral edge. During implantation, Tenon's tissue is drawn forward and stretched to cover the subsidiary ridge of the plate of the implant and sutured firmly to the sclera just in front of the plate. This creates a chamber into which fluid from the anterior chamber of the eye is drained. The tension that the tissue exerts on the subsidiary ridge enables fluid to escape on to the plate beyond the subsidiary ridge only when the pressure reaches a certain level. This reduces the possibility of fluid from the eye being too readily absorbed into the Tenon's tissue overlying the plate in the initial few days after surgery and leading to undesirable excessive IOP reduction.

The plate of the implant described in U.S. Pat. No. 4,750,901 may be linked to one or more other plates with interconnecting tubes to allow passage of fluid from the first (primary) plate to subsidiary plates. This provides a greater surface area for more efficient dispersion of large amounts of aqueous fluid. However, the implantation of more than one plate and tube arrangement greatly increases the complexity and difficulty of surgical implantation. The subsidiary ridge principle described in U.S. Pat. No. 4,750,901 can be applied to any subsidiary plate.

Other glaucoma implants are also known, but these all have problems or disadvantages.

U.S. Pat. No. 5,178,604 (Baerveldt) describes an implant made from a soft elastomeric material for the purpose of avoiding trauma to the eye that can occur with an implant made from a rigid material. However, no such trauma or irritation has ever been observed by the inventor for the present application. Baerveldt claims that a softer material is easier to insert into Tenon's capsule. However, U.S. Pat. No. 6,261,256 (Ahmed) indicates that the opposite may in fact be the case.

U.S. Pat. No. 5,300,020 (L'Esperance, Jr.) describes an implant that is a stud designed to keep a channel open for drainage of aqueous fluid from the eye. However, this implant ignores the need for a plate to be attached to the tubular channel to prevent fibrous tissue capping the end of the channel and preventing the escape of fluid.

U.S. Pat. No. 5,370,607 (Memmen) describes a flexible band and reservoir/tube design with baffles on the surface of the reservoir to direct the flow of fluid.

U.S. Pat. No. 5,397,300 (Baerveldt) describes the use of one or more holes in the plate of the implant described in U.S. Pat. No. 5,178,604. The purpose of the holes is to provide tethering by fibrous tissue designed to stop the bleb distending too much and interfering with the action of the eye muscles and causing double vision (diplopia).

U.S. Pat. No. 5,454,796 (Krupin) describes an implant comprising a tube and a valve within the tube. However, as with all valve designs, there is a risk of blockage by blood clot or debris commonly occurring in the fluid of glaucomatous eyes.

U.S. Pat. No. 5,476,445. (Baerveldt) describes a temporary flow restricting seal beneath the plate of an implant. The implant is designed to provide a temporary, non-valved, flow-restricting device aimed at eliminating post-operative hypotony. The walls of the implant form a seal underneath the plate, between the plate and the sclera, into which the fluid drains. The fluid cannot escape until the pressure within the eye builds up beyond what can be sustained by the sutures holding the plate down onto the sclera The sutures are non-permanent and dissolvable. They anchor the plate to the sclera in the immediate post-operative period thereby preventing post-operative hypotony. The sutures then dissolve such that the plate is anchored only by the downward pressure of Tenon's tissue. This reduces the pressure required to effect fluid release from the anterior chamber. However, the placement of these sutures requires considerable additional surgical time. There is also the danger of fibrous tissue in-growth into the cavity nearest the point where the tube enters from the eye.

U.S. Pat. No. 5,558,629 (Baerveldt) describes an implant similar to the implant of U.S. Pat. No. 5,178,604, as well as additional features including a radio-opaque plate, the use of an absorbable ligature around the tube of the implant, the use of an absorbable plug to restrict the escape of aqueous fluid in the immediate post-operative period, the use of double lumen tubing, and the use of two parallel tubes. This implant would suffer from the same problems experienced by the implant of U.S. Pat. No. 5,476,445 discussed above.

U.S. Pat. No. 5,616,118 (Ahmed) describes an implant having a membrane valve system. The valve opens and closes depending on the intraocular pressure. Like all valved systems it carries the risk of blockage by blood clot or fibrin which would necessitate further surgery or removal of the implant. U.S. Pat. No. 5,681,275 (Ahmed) describes a double plate version and U.S. Pat. No. 5,785,674 (Ahmed) describes a winged version intended to stop strabismus (squinting).

U.S. Pat. No. 6,261,256 (Ahmed) describes an implant having ridges on the surface of the plate to stop it rolling up during insertion. However, in practise, creating multiple blebs by holes in the ridges to provide anchorage for fibrous tissue will tend to reduce the effectiveness of the device and result in higher long term intraocular pressures.

U.S. Pat. No. 6,468,283 (Richter) describes an implant of the stud design. The implant has a small disk at the outer end with a ridge intended to prevent blockage by fibrous tissue. In practise this disk is too small in the vast majority of cases and a cap of fibrous tissue will form over the disk preventing the drainage of fluid.

There is, therefore, a need for further improved implants. In particular, there is demand for a greater drainage area on a single plate to allow for efficient aqueous dispersion while minimising the complexity of surgical implantation. In addition, a lower profile implant is desirable to further assist insertion of the implant into Tenon's capsule. There is also a demand for improved contact between the subsidiary ridge and the overlying Tenon's tissue in order to raise the pressure at which fluid lifts the tissue off the subsidiary ridge and drains onto the larger area of the plate. This would improve the ability of the implant to prevent post-operative hypotony.

Once the implant is in place, the drainage tube is commonly restricted, this is undertaken to prevent conjunctival adhesion and develop an overlying fibrovascular capsule after 4-6 weeks. When the overlying fibrovascular capsule is developed the aqueous humour can then flow freely. Some implants do not come with restriction to the drainage tubes, if the drainage tube is not restricted it can lead to early post-operative hypotony, flat anterior chambers, scaring, and choroidal effusion.

Surgeons may restrict the drainage tube of the implants before use, the drainage tube commonly restricted by a suture tied around the drainage tube. The suture used can be undertaken with non-degradable or degradable sutures. The benefit to the use of a degradable suture is that over time as the suture degrades it allows the aqueous humour to flow freely once it has fully degraded. The duration of the degradation of the sutures is similar to the development of the overlying fibrovascular capsule over the implant, 4-6 weeks. Therefore, once the overlying fibrovascular capsule is developed, the sutures restriction on the drainage tube may be completely absorbed allowing the outflow aqueous humour to flow freely.

Alternatively, a two-stage insertion can be undertaken where the device is sutured at its equatorial position but not connected to the anterior chamber. Once the fibrous chamber has developed only the tube is inserted into the anterior chamber. Between each of the stages, medical therapy or an orphan trabeculectomy is used for IOP control.

Valves have also been used as a form of restriction in the drainage tubes, though studies have shown that the valves added to the total resistance of the aqueous humour flow and can potentially get obstructed in the presence of excess anterior chamber inflammation and fibrin formation. Valves have also been designed to be made of biodegradable materials that degrade after 6 weeks, within the hypertensive phase when the device encapsulation develops. This matches the same time durations as a biodegradable suture on the drainage tube.

Another issue is then a suture is used and applied by a surgeon the strength used to apply the restriction is not always consistent. This can lead to problems with the aqueous humour outflow either being too much or too little.

It is therefore an object of this invention to provide an implant for alleviating problems associated with glaucoma which avoids to at least some extent the problems associated with known implants, or to at least provide a useful alternative to known implants.

### SUMMARY OF THE INVENTION

In a first aspect of the invention there is proposed an eye implant, comprising of a translimbal tube with a restriction applied to it reducing outflow of aqueous humour. The implant and the restriction are then packaged and sterilized together so no further work or adjustments are required by the end user.

In preference the restriction is a knot made of a suture placed around the translimbal tube.

In preference the restriction is in the form of a band around the translimbal tube.

In preference the restriction is in the form of a clip around the translimbal tube.

In preference the restriction is in the form of a cable tie around the translimbal tube.

In preference the restrictions use a degradable material that can be absorbed by a patient.

In a further aspect of the invention there is proposed an ophthalmic implant for treating or alleviating the symptoms of glaucoma, the implant comprising:
a plate shaped to fit a portion of the surface of an eye when implanted, an inner ridge located on the upper convex surface of the plate, where a region encompassed by the inner ridge defines a primary drainage region into which fluid flow from an anterior chamber or posterior chamber of the eye is drawable in use;
a secondary drainage region outside the inner ridge into which fluid from the primary drainage region is receivable in use;
a hole in the inner ridge having a size enabling a drainage tube for draining the fluid from the anterior chamber or posterior chamber of the eye to the primary drainage region to be connected to the hole so that fluid is transferable through the tube and into the primary drainage region, the secondary drainage region is defined by the inner ridge and either the edge of the plate or an outer ridge located on the upper convex surface of the plate, wherein the height of the inner ridge relative to the surface of the plate is greater than the height of the outer ridge relative to the surface of the plate; and
   wherein the drainage tube comprises a fluid flow restriction means.

It should be noted that any one of the aspects mentioned above may include any of the features of any of the other aspects mentioned above and may include any of the features of any of the embodiments described below as appropriate.

The object of this invention is to provide a system and method to address the above shortcomings or at least provides the public with a useful alternative.

### BRIEF DESCRIPTION OF THE DRAWINGS

Preferred features, embodiments and variations of the invention may be discerned from the following Detailed Description which provides sufficient information for those skilled in the art to perform the invention. The Detailed Description is not to be regarded as limiting the scope of the preceding Summary of the Invention in any way. The Detailed Description will make reference to a number of drawings as follows.

FIG. 1 is a perspective view of an eye with an implant of the invention inserted;

FIG. 2 is a side cross-sectional view of an eye with an implant of the invention inserted;

FIG. 3 is a front cross-sectional view through part of the wall of an eyeball in which the implant of the invention has been inserted;

FIG. 4 is a perspective view of the implant of the invention;

FIG. 5 is a perspective view of the translimbal tube with a knot; and

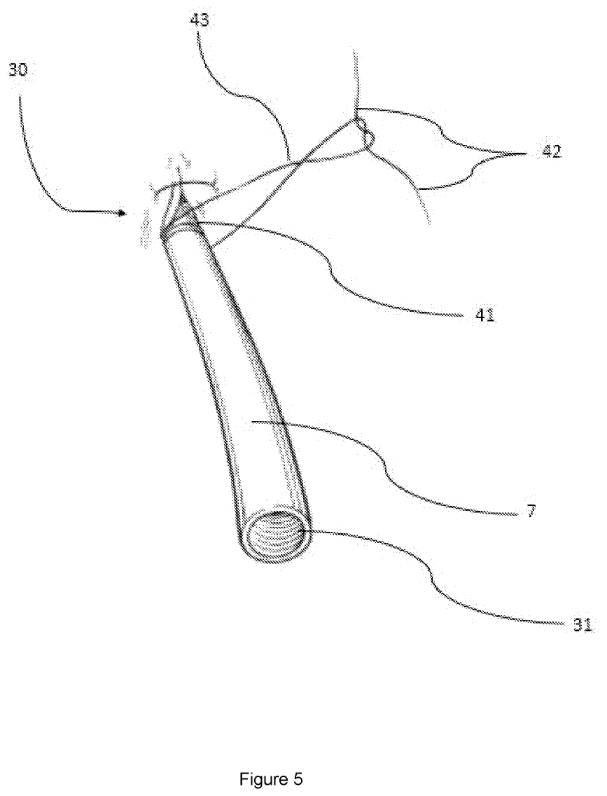

FIG. 6 is a perspective view of the translimbal tube with a band.

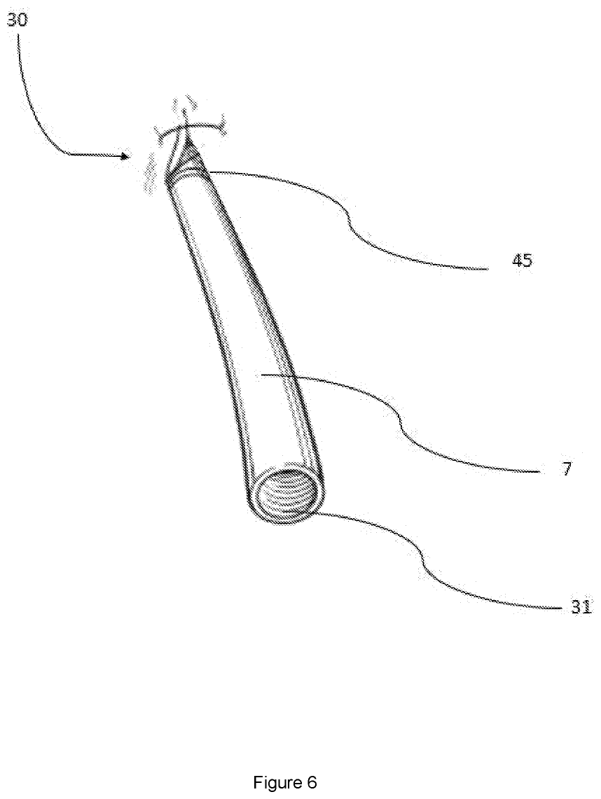

### DETAILED DESCRIPTION OF THE INVENTION

The implant of the invention comprises a plate and tube arrangement of the kind described in U.S. Pat. No. 4,457,757. IOP is reduced by the transfer of aqueous fluid from the interior of the eyeball through the tube to the plate. Fluid that is transferred to the plate is absorbed into surrounding tissues. Preferably, the tube is inserted through the sclera and into the anterior chamber or the posterior chamber of the eye, thus providing fluid communication between the interior of the eye and the surface of the plate.

The present invention also utilises a ridge mechanism as described in U.S. Pat. No. 4,750,901 in order to prevent post-operative hypotony. The ridge mechanism comprises an inner ridge defining a part of the surface area of the plate and an outer ridge at or near to the edge of the plate. However, the ridge mechanism of U.S. Pat. No. 4,750,901 has been improved in this invention by providing an improved connection between the inner ridge and the overlying Tenon's tissue.

The preferred implant of this invention has an enlarged plate relative to some other implants. This provides a greater fluid dispersion area. The need for multiple plate implants is therefore reduced, and the surgical problems associated with multiple plate implants avoided.

The preferred implant of this invention also has either no outer ridge at or near to the edge of the plate or has a low-profile outer ridge. It has been found that an outer ridge is not essential to maintain a bleb over the plate. The reduced outer ridge lowers the profile of the implant making it easier to insert into the Tenon's capsule.

The enlarged plate together with the lower outer ridge of the implant of this invention also helps to improve contact between the inner ridge and the overlying Tenon's tissue. The increased width of the plate allows the implant to be positioned so that at least a portion of each of two opposite sides of the plate lies beneath a rectus muscle insertion. Tenon's tissue that is adjacent to a rectus muscle insertion is reinforced by fibrous expansions which extend from the rectus muscle sheath into Tenon's tissue. This allows Tenon's tissue to be drawn more tightly across the inner ridge during implantation. Consequently, the fluid pressure required to lift Tenon's tissue off the inner ridge to allow fluid to drain from the region encompassed by the inner ridge (the primary drainage region) onto the larger area of the plate beyond the inner ridge (the secondary drainage region) is raised and the ability of the implant to prevent post-operative hypotony is thereby improved.

Enzymes produced by the tissues covering the implant act to remove blood clot or inflammatory exudates that can cause blockage of valve-based implants, stud implants, or implants where the drainage tube is located underneath the plate. In addition, any blockage by blood clot or inflammatory exudate that does form is rapidly cleared by enzymes from the overlying tissue. This is not the case with implants with mechanical valves, such as the Krupin and the Ahmed inventions where blockage may require repeated surgery or removal of the implant.

The implant of the invention may be made of any biocompatible material, rigid or flexible. Preferably, the implant is made of polypropylene. In a preferred embodiment, the implant is quite flexible on account of its thinness and the lack of a pronounced outer ridge.

The plate of the implant is curved to fit the curvature of the eyeball. The plate preferably has holes near the edge of the plate which allow sutures to be threaded through the plate for the purpose of attaching the plate to the surface of the eyeball. In a preferred embodiment the plate has four suture holes.

The plate is attached to the surface of the eyeball under the outer tissue layer which is known as Tenon's tissue. At operation, the plate is inserted between the sclera and Tenon's capsule and sutured to the surface of the eyeball (the sclera).

The implant may be positioned in any of the four quadrants of the eye, the superior temporal and superior nasal quadrants being the most commonly used.

Preferably, the plate is positioned so that it lies between the tendon insertions of two adjacent rectus muscles on the surface of the eyeball. The plate is held in place by the normal muscle tone of the adjacent rectus muscles and by sutures which pass through the anterior suture holes of the plate and the tendon insertions of the rectus muscles.

The dimensions of the implant are variable. Preferably however, the width of the plate is such that the sides of the plate can be inserted partly beneath adjacent rectus muscles. Preferably, the length of the plate is such that it avoids the optic nerve and the blood vessels at the back of the eye. In a preferred embodiment, the plate is designed to fit between and slightly under the edge of each of two adjacent rectus muscles.

The plate of the implant may be linked to one or more other plates with interconnecting tubes to allow passage of fluid from the first (primary) plate to subsidiary plates. However, in a preferred embodiment of the invention, the implant has a plate of sufficient surface area to eliminate the need for multiple plate implants in most cases.

An outer ridge may be present with a low profile or may be absent from the surface of the plate. The applicant has found that a pronounced outer ridge is not necessary for bleb formation and maintenance. In a preferred embodiment, the outer ridge is significantly reduced in size relative to the outer ridge described in U.S. Pat. No. 4,750,901. Reduction or removal of the outer ridge provides an implant having a reduced profile which is therefore easier to insert into Tenon's capsule.

As noted above, a problem associated with implants of this kind is an excessive lowering of IOP following implantation (post-operative hypotony). This can cause damage to the eye resulting in impairment of vision.

This problem is avoided in the present invention by utilising a ridge mechanism as described in U.S. Pat. No. 4,750,901. However, the ridge mechanism of U.S. Pat. No. 4,750,901 has been improved in this invention by providing an improved connection between the inner ridge and the overlying Tenon's tissue.

The inner ridge incorporates the opening of the tube onto the upper surface of the plate.

The size of the region defined by the inner ridge (the primary drainage region) may vary from an area just big enough to incorporate the opening of the tube where it emerges onto the surface of the plate, to an area that occupies a large proportion of the plate. However, the larger the primary drainage region, the lower the pressure at which fluid can escape into the secondary drainage region. In a preferred embodiment, the primary drainage region occupies less than one quarter of the surface area of the plate.

The implant may have additional ridges each of which define a further ridged area at different locations on the surface of the plate. The ridge principle of this invention may also be applied to any subsidiary plate arrangements.

During implantation, Tenon's tissue is drawn forward and stretched to cover the inner ridge of the plate of the implant and sutured firmly to the sclera just in front of the plate. This creates a chamber into which fluid from the anterior chamber or posterior chamber of the eye is drained. The tension that the tissue exerts on the inner ridge enables fluid to escape on to the plate beyond the inner ridge, to the secondary drainage region, only when the pressure reaches a certain level.

When fluid begins to flow through the tube of the implant, Tenon's tissue, stretched over the inner ridge of the implant, provides sufficient resistance to the escape of fluid from the eye to limit drainage of fluid to the primary drainage region. This reduces the possibility of fluid from the eye being too readily absorbed into the Tenon's tissue overlying the larger area of entire plate in the initial few days after surgery and therefore avoids post-operative hypotony. When the IOP rises to a sufficient level to lift Tenon's tissue off the inner ridge, drainage through the entire area of Tenon's tissue overlying the plate of the implant commences and the IOP falls gradually. If the IOP reduces below the above level, the Tenon's tissue will return to contact with the inner ridge to reduce or prevent flow of fluid into the secondary drainage region. In this way, the inner ridge in combination with the Tenon's tissue acts as a valve.

The thickness of Tenon's tissue varies. It is greatest between the equator of the eye and the tendon insertions of the muscles. In a preferred embodiment of the invention, it is this thicker portion of Tenon's tissue that is stretched over the inner ridge of the implant at operation. Manipulation of the tissues at the time of operation, together with the pro-inflammatory effect of the fluid on the tissues, raises the pressure in Tenon's tissue to about 12 to 15 mmHg. Preferably, IOP must be more than about 12 to 15 mmHg before Tenon's tissue is lifted off the inner ridge of the implant to allow drainage of fluid into the secondary drainage region.

An absorbable ligature may or may not be used to delay drainage of fluid in appropriate cases.

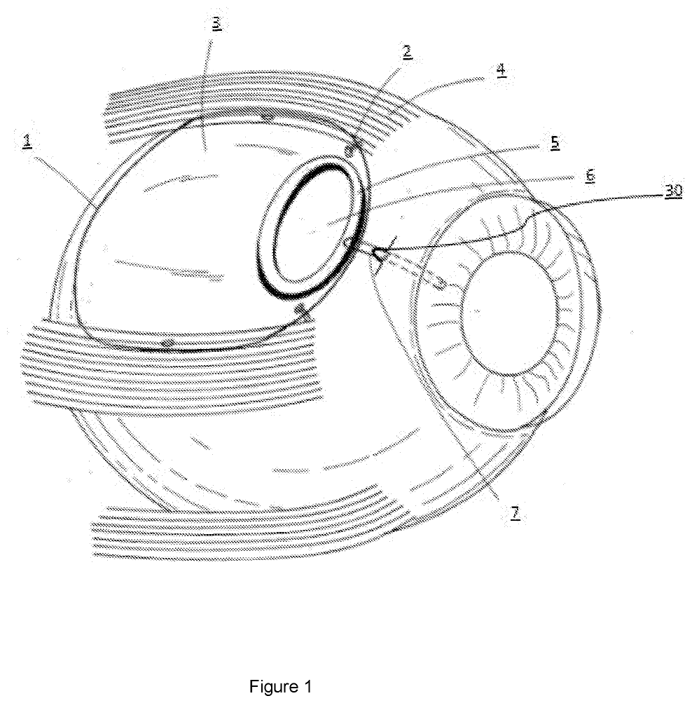

The invention will now be further described by way of example only with reference to FIGS. 1 to 4 of the drawings.

FIG. 1 shows the implant 1 of the invention attached to the surface of an eyeball by sutures 2 threaded through holes positioned near the edge of the implant plate 3. The plate 3 is positioned between the tendon insertions of two rectus muscles 4 so that a portion of each of two opposite sides of the plate 3 lies beneath a rectus muscle insertion.

The plate 3 is curved to fit the curvature of the eye and to enable the plate 3 to sit neatly on the surface of the eye.

An oval shaped inner ridge 5 extends from the upper (convex) surface of the plate 3 at the front edge of the plate 3 (in relation to the eye). The inner ridge 5 forms a primary drainage region 6 on the upper surface of the plate 3.

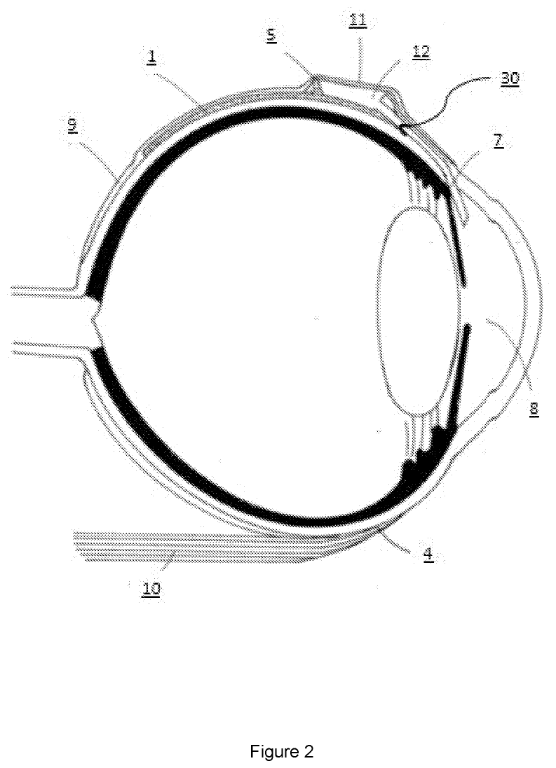

A translimbal tube 7 extends from the region 6, through the limbal tissue, into the anterior chamber 8 of the eye, as shown in FIG. 2. The translimbal tube 7 enables the transfer of fluid from the anterior chamber 8 of the eye into the region 6, thereby reducing pressure within the anterior chamber 8.

As shown in FIG. 2, the implant 1 is located on the upper surface of the eye beneath the outer tissue layer of the eyeball (Tenon's capsule 9). The rectus muscle 10 is in contact with the eyeball from the equator of the eyeball to the point at which the rectus muscle 10 is anchored to the anterior surface of the eyeball (the rectus muscle tendon insertion 4). Posterior Tenon's tissue is relatively thin. However, anterior Tenon's tissue is thicker due to reinforcement by fibrous expansions that extend from the rectus muscle sheaths. This reinforced thicker anterior portion of Tenon's tissue 11 covers the region 6 and is held in contact with the inner ridge 5 and stretched over it by the natural muscle tone of the adjacent rectus muscles 4. This helps to maintain contact between the inner ridge 5 and the overlying tissue 11.

FIG. 2 further shows that Tenon's tissue forms a layer over the region 6 on the upper surface of the plate 3, thereby defining a chamber 12. The chamber 12 is capable of holding fluid transferred from the anterior chamber 8 of the eye through the tube 7.

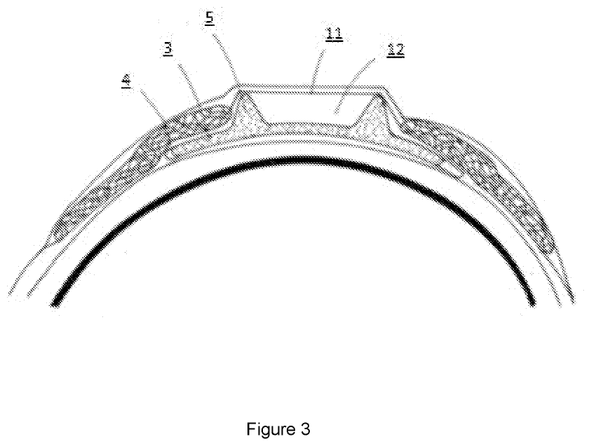

The chamber 12 is further illustrated in FIG. 3. FIG. 3 also illustrates the sides of the plate 3 immediately behind a rectus muscle insertion 4 and the Tenon's tissue that forms a layer over the inner ridge 5 thickened by fibrous expansions from the rectus muscle sheaths and held in contact with the inner ridge 5 by the tone of the muscles.

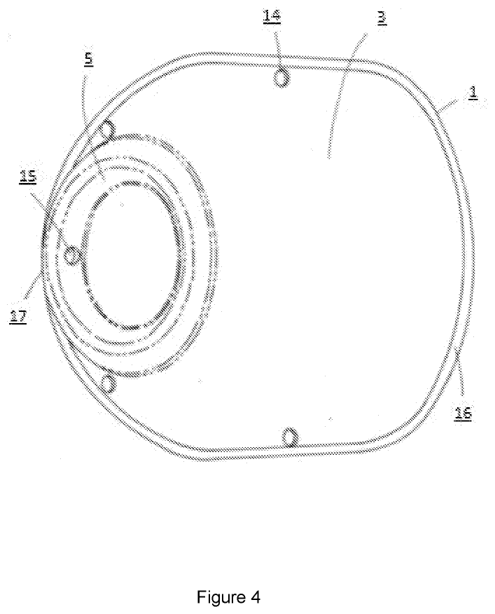

FIG. 4 shows that the plate 3 has a rounded shape with curved front and rear ends but straighter sides, and comprises four holes 14 located near the edge of the plate 3. The holes 14 allow the plate 3 to be sutured to the surface of an eyeball.

The inner ridge 5 extends from the surface of the plate 3 near the front end 17 of the plate 3. The portion of the inner ridge 5 closest to the periphery of the plate 3 has a hole 15. The end of a translimbal tube (not shown in FIG. 4) fits snugly into the hole 15 from the exterior of the inner ridge 5 and leads to the anterior chamber of the eye when the implant 1 is surgically implanted. An outer ridge 16 is located at the edge of the plate 3 and merges with the ridge 5 at the front end 17 of the plate 3.

The invention therefore provides an implant that provides a greater fluid dispersion area within a single plate and tube device, a lower profile for easier implantation, and improved contact between the inner ridge and the overlying Tenon's tissue. The implant of the invention therefore improves the ability of the implant to prevent post-operative hypotony. The plate, together with the living tissue covering it, form a biological tissue/plate valve which regulates the escape of fluid from the eye in the early postoperative period. Unlike valve-based implants, stud implants, or implants wherein the drainage tube is located underneath the plate which can become blocked by a blood clot or inflammatory exudates, this biological valve system ensures that the drainage system cannot become blocked by blood clot or inflammatory exudates.

A restraint 30 can be placed around the translimbal tube 7 to control the intraocular pressure (IOP) and reduce the flow of the aqueous humour. The restraint 30 can be in the form of but not limited to a knot 41, band 42, partially closed valve, clamp, cable tie, and insertion tube. The preference is to use a knot 41 made with a suture 43 around the translimbal tube 7.

A fluid flow control mechanism may comprise a biodegradable material fully or partially occluding the drainage tube.

A drainage restriction may be made of a drug eluting material, which may elute active pharmaceutical ingredients such as common anti-inflammatory (salicylates, prostaglandin antagonists, leukotriene antagonists, polyphenols, and vasoconstrictors), IOP reducing (topical β-adrenergic antagonists, carbonic anhydrase inhibitors, cholinergics, α-adrenergic agonists prostaglandins or corticosteroids (Dexamethasone, hydrocortisone, methylprednisolone and prednisone) used postoperatively following GDD implantation.

There are no limitations to the number of restraints 30 used on the translimbal tube 7 at the same time, it can preferably range from 1-3 restraints 30.

The restraint 30 is used to reduce the drainage through the translimbal tube 7 of the aqueous humour from the anterior chamber 8.

The knot 41 restricts 30 the fluid flow in the translimbal tube 7 by tightening around the outer surface of the translimbal tube 7 and reducing the inner diameter of the trans limbal inner orifice 31. As the inner diameter is reduced the fluid flow from the anterior chamber 8 through the translimbal tube 7 is reduced. The implant 1 translimbal tube 7 has an outer diameter of but not limited to 0.63 mm and a bore of 0.30 mm. After the knot 41 is applied, the internal diameter 31 of the translimbal tube 7 can be between but not limited to 0.01 to 29 mm.

The knot 41 is commonly tied with a suture 43, the type of suture 43 can be but not limited to a 6-0 polypropylene suture, 5-0 coated vicryl suture, and a monofilament poliglecaprone 25 suture. Other types of material for the sutures can also be used such as but not limited to Monocryl, Caprosyn, Vicryl, Polysorb, Dexon, PDS, Biosyn, Maxon, and Nylon.

The material of preference for the knot 41 restrictions can be made of but not limited to polypropylene. The benefit of the material is that it can be autoclaved, gamma sterilized, ETO and various sterilization methods, subject to the survivability of the chosen material along with the implant. The benefit of having the implant 1 and knot 18 sterilised at the same time ensures that the device can be used without further requirements for sterilisation. The sterilisation can be undertaken in the final packaging for which the user will receive, therefore once the user receives the implant 1 there is no further requirement for the user to undertake.

The type of knots 41 that are used can be but are not limited to square knot, clove-hitch, constrictor, ligature, surgical tie, granny knot, or surgeons knot.

The knot 41 flow rate can be tested by injecting saline via closely fitting a syringe to the translimbal tube 7 and applying pressure with a saline solution.

A poliglecaprone suture tensile strength reduces over time. As an example, but not limited to a poliglecaprone suture will lose 50-60% of its original tensile strength after 7 days, 20-30% of its original tensile strength after 14 days and absorbed after 90-120 days. This provides a variable flow rate over time for the translimbal tube 7 for the aqueous humour. As the suture 43 slowly dissolves away over time the tensile strength of the knot 41 on the translimbal tube 7 is reduced, allowing the internal diameter 31 of the translimbal tube 7 to increase back to its original size and allowing the aqueous humour to flow freely.

When the suture 43 is fully dissolved the translimbal tube 7 can flow freely allowing the IOP to return to normal. If a non-dissolvable suture 43 is used, it will need to be removed after a selected period of time.

The pressure of the knot 41 on the translimbal tube 7 can be varied to change the internal diameter of the translimbal tube 7. The pressure of the knot 41 on the translimbal tube 7 can range from but is not limited to 1 to 200 mmHg. To achieve a variation in pressure the knot 41 will be placed around the tube and tightened by pulling the knots ends 42 until the desired tension is achieved. This pressure can be measured with strain gauges on the devices that are pulling the knot ends 42.

After the knot 41 is placed around the translimbal tube 7, the knot ends 42 are cut off leaving but not limited to 2-3 mm of suture remaining.

The knot 41 can be applied to the translimbal tube 7 either by but not limited to manually or mechanical devices. The use of a machine or mechanical devices to tie the knot 41 around the translimbal tube 7 ensures that a desired amount of torque is obtained on the knot 41. A machine or mechanical means to tie the knot 41 can ensure there is consistency achieved with the amount of torque.

The translimbal tube 7 can also be restricted by placing a band 45 around the translimbal tube 7. The band 45 is placed onto the translimbal tube 7 squeezing the tube 7 and reducing the inner diameter, in turn reducing the potential outflow of the aqueous humour.

The position of the restriction can be located anywhere on the translimbal tube 7. For example, the restriction can be located close to the implant 1 or near the end of the translimbal tube 7.

A means to determine the potential pressure in the translimbal tube 7 can be in the form of a band 45 placed around the tube that changes colour or the distance between know points on the band shift a certain distance. For example, as the pressure increases in the translimbal tube 7, the bands 45 colour will become lighter since the outer surface is stretched further apart allowing the inner surface which is a lighter colour to be visible. Though as the pressure decreases the colour becomes darker. Also, points on the bands 45 can be marked, as the pressure increases the two points will be shifted further apart, the distance can be measured between the two points to determine the internal pressure in the translimbal tube 7.

A mechanical means can also be used to restrict the translimbal tube, various forms of mechanical restriction such as a but not limited to a cable tie or clamp. The cable tie (not shown) or clamp (not shown) can be in the form of a degradable or non-gradable material.

A cable tie (not shown) to a set diameter that corresponds to a desired reduced flow rate can be placed around the translimbal tube 7 and pulled until it reaches a set point.

A clamp (not shown) can also be used to reduce the flow in the translimbal tube 7. The clamp can have a set diameter that corresponds with a desired reduced flow rate. The translimbal tube 7 is placed into the clamp, the clamp is then fastened around the translimbal tube 7 with a clip. When the clamp is closed around the translimbal tube 7, the translimbal tube 7 will be compressed and the aqueous humour outflow will be reduced.

Another means of restricting the tube can be in the form of inserting another tube (not shown) into the open end of the translimbal tube 7. The restricting tube can have an outer diameter that will match the internal diameter of the translimbal tube 7 and has an internal diameter that will restrict the flow to the desired flow rate. The tube can be removed from the end of the translimbal tube 7 and a new tube with the desired inner diameter inserted.

After the restraint 30 has been placed onto the translimbal tube 7, the implant 1 along with the restraint 30 are packed into a container and then sterilised. This then allows the implant 1 and the restraint 30 to be ready for use without any further amendments required by the users prior to application in a patient.

The forms of sterilisation used can be in the form of but not limited to an autoclave or gamma irradiation. Providing an implant 1 to the user that is ready for application to the patient with a restriction in place prevents any potential for contamination that could potentially be introduced. The user can simply open the packaging and use the implant 1.

Although the invention has been described by way of example, it should be appreciated that variations and modifications may be made without departing from the scope of the claims. Furthermore, where known equivalents exist to specific features, such equivalents are incorporated as if specifically referred in this specification.

### INDUSTRIAL APPLICABILITY

The ophthalmic implant of the invention is useful for treating or alleviating the symptoms of glaucoma. Following surgical implantation, the implant (with drainage tube attached) enables fluid to be transferred from within the eye to the upper surface of the implant's plate and from there the fluid is absorbed into surrounding tissue. Intraocular pressure is thereby reduced and the effects of glaucoma alleviated.

### LIST OF COMPONENTS

The drawings include the following integers.
1 Implant
2 Sutures
3 Implant plate
4 Insertion
5 Inner ridge
6 Drainage region
7 Translimbal tube
8 Anterior chamber
9 Tenon's capsule
10 Rectus muscle
11 Overlying tissue
12 Chamber
14 Plate holes
15 Holes
16 Outer ridge
17 Front end of the plate
30 Restraint
31 Internal orifice
41 Knot
42 Knot ends
43 Suture
45 Band

Further advantages and improvements may very well be made to the present invention without deviating from its scope. Although the invention has been shown and described in what is conceived to be the most practical and preferred embodiment, it is recognized that departures may be made therefrom within the scope of the invention, which is not to be limited to the details disclosed herein but is to be accorded the full scope of the claims so as to embrace any and all equivalent devices and apparatus. Any discussion of the prior art throughout the specification should in no way be considered as an admission that such prior art is widely known or forms part of the common general knowledge in this field.

In the present specification and claims (if any), the word “comprising” and its derivatives including “comprises” and “comprise” include each of the stated integers but does not exclude the inclusion of one or more further integers.

1. An ophthalmic implant for treating or alleviating the symptoms of glaucoma, the implant comprising:
a. a plate shaped to fit a portion of the surface of an eye when implanted,
an inner ridge located on the upper convex surface of the plate, where a region encompassed by the inner ridge defines a primary drainage region into which fluid flow from an anterior chamber or posterior chamber of the eye is drawable in use;
b. a secondary drainage region outside the inner ridge into which fluid from the primary drainage region is receivable in use;
c. a hole in the inner ridge having a size enabling a drainage tube for draining the fluid from the anterior chamber or posterior chamber of the eye to the primary drainage region to be connected to the hole so that fluid is transferable through the tube and into the primary drainage region, the secondary drainage region is defined by the inner ridge and either the edge of the plate or an outer ridge located on the upper convex surface of the plate, wherein the height of the inner ridge relative to the surface of the plate is greater than the height of the outer ridge relative to the surface of the plate; and
d. wherein the drainage tube comprises a fluid flow restriction means.

2. The ophthalmic implant as in claim 1 wherein the fluid flow restriction means occludes the drainage tube.

3. The ophthalmic implant as in claim 1, wherein the fluid flow restriction on the drainage tube is comprised of a knot.

4. The knot as in claim 3, wherein the knot is made of a degradable suture.

5. The ophthalmic implant as in claim 1, wherein the fluid flow restriction on the drainage tube is comprised of a band.

6. The band as in claim 5, wherein the band is degradable.

7. The ophthalmic implant as in claim 1, wherein the fluid flow restriction means is comprised of a clip.

8. The band as in claim 7, wherein the clip is degradable.

9. The ophthalmic implant as in claim 1, wherein the fluid flow restriction means is comprised of a cable tie.

10. The cable tie as in claim 9, wherein the cable tie is degradable.

11. The ophthalmic implant as in claim 1, wherein it is autoclaved or sterilised with the fluid flow restriction means attached.
